(19) United States
(12) Patent Application Publication
(10) Pub. No.: US 2020/0082932 A1
(43) Pub. Date: Mar. 12, 2020

(54) Patient Engagement Plan Generation and Implementation Based on Program Specific Factors

(71) Applicant: International Business Machines Corporation, Armonk, NY (US)

(72) Inventors: Marc Salinas, Prosper, TX (US); Joshua P. Schiffman, New York, NY (US)

(21) Appl. No.: 16/123,224

(22) Filed: Sep. 6, 2018

(51) Int. Cl. G16H 40/20; G16H 80/00; G16H 20/60; G06Q 30/02; G16H 50/30; G16H 10/60

(52) U.S. Cl. CPC G16H 40/20; G16H 80/00; G16H 10/60; G06Q 30/0283; G16H 50/30; G16H 20/60

(57) ABSTRACT

Mechanisms are provided to implement an engagement optimization engine for generating and implementing a personalized patient engagement plan to monitor a patient's adherence to a personalized patient care plan. The engagement optimization engine receives a cohort of patients, each having different personal characteristics. For each patient, the engagement optimization engine identifies a set of patient-specific factors and a set of program-specific factors associated with a set of programs available to the patient. The engagement optimization engine evaluates each set of patient-specific factors and the program-specific factors to generate the personalized patient engagement plan for each patient to meet the needs of the patient. The engagement optimization engine outputs the personalized patient engagement plan for each patient to an assessor in order to implement monitoring of the personalized patient care plan generated for the patient.

### BACKGROUND

The present application relates generally to an improved data processing apparatus and method and more specifically to mechanisms for generating and implementing a patient engagement plan based on program specific factors.

Monitoring patients with chronic illnesses, such as congestive heart failure, diabetes, and asthma represents one of the greatest challenges facing modern medicine. Patients with chronic illnesses require ongoing, follow-up treatment and care to properly manage their conditions. Unfortunately, a number of these patients do not receive ongoing treatment and care, receive treatment and care on a sporadic basis, or receive treatment and care which is not in accordance with recommended guidelines. Worse, patients often fail to do the basic simple day-to-day tasks that could prevent or reduce the frequency and magnitude of a catastrophic event such as a hospitalization. As a result, these patients often unnecessarily suffer from symptoms of their chronic illness which would have been minimized or prevented with proper ongoing treatment and care. Additionally, some of these patients may later require hospitalization, or in severe cases some of these patients may die, both of which may have been prevented if the patient was receiving the proper ongoing treatment and care.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising a processor and a memory, the memory comprising instructions that are executed by the processor to configure the processor to implement an engagement optimization engine for generating and implementing a personalized patient engagement plan to monitor a patient's adherence to a personalized patient care plan. The method comprises receiving, by the engagement optimization engine, a cohort of patients with each patient in the cohort of patients having different personal characteristics. The method also comprises identifying, by the engagement optimization engine, a set of patient-specific factors for each patient in the cohort of patients. The method further comprises identifying, by the engagement optimization engine, a set of program-specific factors associated with a set of programs available to the patient. Moreover the method comprises evaluating, by the engagement optimization engine, each set of patient-specific factors and the program-specific factors to generate the personalized patient engagement plan for each patient such that the personalized patient engagement plan meets the needs of the patient in view of the set of programs offered. Additionally, the method comprises outputting, by the engagement optimization engine, the personalized patient engagement plan for each patient to an assessor in order to implement monitoring of the personalized patient care plan generated for the patient.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is a block diagram illustrating a cloud computing system 100 for providing software as a service, where a server provides applications and stores data for multiple clients in databases according to one example embodiment of the invention;

FIG. 2 is another perspective of an illustrative cloud computing environment in which aspects of the illustrative embodiments may be implemented;

FIG. 3 is an example diagram illustrating a set of functional abstraction layers provided by a cloud computing environment in accordance with one illustrative embodiment;

FIG. 4 is an example block diagram illustrating the primary operational elements of such a personalized patient care plan (PPCP) creation and monitoring system in accordance with one illustrative embodiment;

FIG. 5 is a flowchart outlining an example operation for creating a personalized patient care plan (PPCP) in accordance with one illustrative embodiment.

FIG. 6 is a flowchart outlining an example operation for generating a personalized patient engagement plan (PPEP) in accordance with one illustrative embodiment;

FIG. 7 is a flowchart outlining an example operation for monitoring a patient's performance with regard to a prescribed personalized patient care plan (PPCP) based on the personalized patient engagement plan (PPEP) in accordance with one illustrative embodiment; and

FIG. 8 is a flowchart outlining an example operation for adjusting a personalized patient health care plan based on an evaluation of a patient's adherence to a prescribed personalized patient health care plan in accordance with one illustrative embodiment.

### DETAILED DESCRIPTION

Before beginning the discussion of the various aspects of the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In the following description, reference is made to embodiments of the invention. However, it should be understood that the invention is not limited to specific described embodiments. Instead, any combination of the following features and elements, whether related to different embodiments or not, is contemplated to implement and practice the invention. Furthermore, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

In addition, it should be appreciated that the present description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

As noted above, providing treatment and care for patients having illness requiring ongoing treatment is a major issue in modern medicine. Many times this ongoing treatment and care is a shared responsibility between the medical workers, e.g., doctors, nurses, etc. and the patient. That is, each patient must perform certain actions on their own to provide self-treatment for the illness, which often involves making different lifestyle choices, e.g., changing diet, increasing physical activity, taking prescribed medications, eliminating habits and consumption of products that are detrimental to health, etc., with the medical workers providing monitoring and periodic checks of the patient's progress to ensure that the patient is adhering to the treatment needed to control and/or improve the patient's condition.

A number of mechanisms have been developed for assisting the patients and medical workers in handling their shared responsibilities including mechanisms for generating patient care plans based on the patient's medical condition, mechanisms for patients to self-monitor their adherence to their own care plans, and the like. Such mechanisms often regard patients as generic types of patients, e.g., a generic asthma patient, a generic diabetes patient, etc. possibly with some classification within these generic categories based on the patient's age, gender, race, and other generic demographics. Even with such classification within the generic categories, the resulting care plan associated with the patient is one that is applicable to multiple patients having the same set of medical diagnosis and demographics. The care plan is not in fact personalized to the specific patient but to a general categorization of the patient.

Each individual patient has a specific and different set of lifestyle conditions that make that patient unique from other patients. It is this uniqueness that is not reflected in the patient care plans generated by known mechanisms.

That is, the known patient care plan mechanisms are created to classify patients into generic categories and apply generic care plans to these patients. While mechanisms employing such patient care plan mechanisms may refer to them as being “personalized” or “customized” to the patient, they in fact are only superficially customized in that they may be customized based on generic customization categories, e.g., customized based on generic demographics such as age, race, gender, etc. As a result, patients are not in fact presented with a patient care plan that the patient feels is specifically suited to them. The patient care plans do not in fact take into account the patient's own individual circumstances and can be applied to a plurality of patients having the same demographics and medical condition, e.g., all 40 year old female diabetes patients. There are no mechanisms that personalize a patient's on-going treatment and care based on both their medical condition and the patient's own personal lifestyle, taking into account multiple lifestyle conditions and the facilities and resources available to that particular patient based on their lifestyle.

It should be appreciated that the term “lifestyle” as it is used herein refers to the way in which a person lives their lives. The term “lifestyle information” refers to the data collected that characterizes the lifestyle of the patient and may encompass various temporal, spatial, environmental, and behavioral information/data about the patient that together comprises a unique combination of information/data that characterizes and represents the way in which that specific patient conducts their life on a daily basis. The lifestyle information for a patient is specific to that patient and is not generally applicable to multiple patients. The lifestyle information may be provided at various levels of granularity depending upon the particular implementation. As part of this lifestyle information, data generated by the specific patient via one or more computing devices or other data communication devices may be included such as actions performed by the patient on a daily basis, personal schedules, specifications of preferences, etc. For example, lifestyle information may include the patient entering information, such as into a computing device executing a patient tracking application, indicating that the patient ate breakfast at a fast food restaurant in the airport on the way to Virginia this morning. In addition, data generated by external systems associated with third parties that characterizes the patient's lifestyle may be included in the lifestyle information as well, e.g., a healthcare insurance company may have information about the patient's lifestyle, e.g., smoker, overweight, sedentary, high risk for diabetes, etc., which may be characteristic of the patient's lifestyle.

For example, with regard to temporal lifestyle information, the lifestyle information may comprise one or more data structures specifying one or more schedules of events that the patient undergoes either on a routine basis or on a dynamic basis, e.g., a baseline routine schedule that may be dynamically updated as events occur or do not occur. The temporal lifestyle information may comprise, for example, the time that the patient wakes in the morning, when they have their meals, when they go to work and return home, when they take their children to school, when they shop for groceries, when they go to bed at night, scheduled non-routine events, free time, scheduled flight, ferry, train, or other ground transportation departure/arrival times, and/or any other temporal information characteristic of the patient's daily life and other non-routine scheduled events.

With regard to spatial lifestyle information, this information may comprise one or more data structures identifying locations associated with the patient's daily lifestyle including routine locations frequented by the patient, e.g., the location of their home, the location of their work, the location of their child's school, the location of the retail establishments that they frequent, the location of their doctors, the typical travel paths between locations utilized by the patient, and the like. The spatial lifestyle information may further comprise information about each location including the number of stories or levels in the buildings, e.g., two-story home, five-story office building, etc., whether the location has stairs, etc. The spatial lifestyle information may further comprise geographic information including the city, state, county, country, etc., in which the patient lives, works, travels to, or otherwise conducts their life.

With regard to environmental lifestyle information, this information comprises one or more data structures with indications of the environmental quality and resource availability in the environments in which the patient is present, is predicted to be present at a later time (such as based on the temporal and spatial lifestyle information), or typically is present on a daily or routine basis. For example, environmental lifestyle information may include information about the patient's home location, e.g., in a rural, urban, or suburban environment, has access to parks, walking trails, etc. This environmental lifestyle information may include information about the patient's work location including whether the patient works in an office setting with fluorescent lights and relative quiet, in a manufacturing setting with heavy machinery and loud noises, works with computers the majority of the day, has his/her own office or is in a cubicle, the number of co-workers the patient has that they interface with on a daily basis, the types and/or identities of establishments around the patient's home/work for purposes of determining access to resources (e.g., products and services), air quality, weather conditions, elevation (for purposes of oxygen level determination, for example), and the like.

Regarding behavioral lifestyle information, this information comprises one or more data structures having indications of the patient's own behavior and likes/dislikes, i.e. lifestyle preferences. The behavioral lifestyle information may comprise such information as the patient's habits, responses to communications of different modalities, patterns of activity, and the like. For example, such behavioral lifestyle information may indicate that the patient has a habit of eating a snack every evening after 9 p.m. or takes his/her dog for a walk in the mornings before 9 a.m. and after 5 p.m. The behavioral lifestyle information may further indicate the patient's likes and dislikes (preferences) with regard to various elements of daily life including types of foods the patient likes/dislikes, types of physical activity the patient likes/dislikes, when the patient likes to engage in certain activities, e.g., exercising before work/after work, or the like.

The various lifestyle information data may be obtained directly from the patient, such as via an electronic questionnaire, through analysis of electronic medical records (EMRs) or other entries in databases associated with the patient (e.g., governmental databases associated with a patient's social security number, address, or the like), or otherwise obtained from one or more monitoring devices and/or applications utilized on one or more computing devices associated with the patient and with which the patient interacts, e.g., patient tracking applications on a smart phone, a medical monitoring device, or the like, that monitors physical activity, food logs, and the like. This lifestyle information may be generated from static information and may also be dynamically updated on a periodic or constant basis to obtain the most current lifestyle information representative of the patient's current lifestyle. The lifestyle information is utilized to customize or personalize a patient care plan for the specific patient such that the patient is presented with a resulting patient care plan that the patient feels is tailored specifically to them and they way they conduct their lives.

In addition to known patient care plan mechanism suffering from the drawback of not in fact generating personalized patient care plans (PPCPs) taking into account a patient's unique lifestyle, the known patient care plan mechanisms also do not provide for the ability to integrate third-party information about the lifestyle of a patient into the patient care plan personalization such that a more complete understanding of the capabilities of the patient based on their lifestyle is realized when generating and monitoring the patient's adherence to the patient care plan. For example, third-party lifestyle information may comprise information from commercial and governmental computing systems, databases, and the like, that characterize the patient's environment, availability to resources (e.g., products/services/facilities), etc., or is otherwise ancillary and further defining of other lifestyle information associated with the patient.

As one example, a third-party lifestyle information source may comprise a global positioning system (GPS) source that identifies the patient's associated locations, e.g., home, work, etc., and identifies establishments around those locations that provide resources that are of interest to the patient's lifestyle and potentially of interest in generating a patient care plan. For example, specialty grocery stores, vitamin stores, pharmacies, restaurants, gyms, walking paths, parks, recreational areas, community pools, and the like, may be identified based on a GPS system and its associated databases of information. This information may include identifications of types (e.g., Vietnamese Restaurant) and specific identities (e.g., “Fabulous Pho”) of the particular establishments which can be used with other third-party lifestyle information sources (e.g., “Fabulous Pho” website comprising menu and nutrition information) to retrieve specific information about those identified establishments. For example, a particular restaurant may be determined to be within a specified distance of the patient's home location and corresponding restaurant menu item information and hours of operation information may be retrieved from that particular restaurant's website, computing system, or other database. The retrieved menu item information and hours of operation information may be used, as described hereafter, to correlate the information with patient care plan information, e.g., nutritional and caloric information may be correlated with the patient care plan, to generate patient care plan actions/tasks and/or recommendations for assisting the patient in adhering to the patient's PPCP. Similarly, other third-party lifestyle information sources may provide information for correlation with patient care plan actions/tasks including hours of operations, products/services provided, distance from the patient's locations, and the like.

The illustrative embodiments of the present invention collect patient demographic and medical data, such as from questionnaires, electronic medical records, and the like, and generate a baseline patient care plan based on an initial diagnosis of the patient's medical condition, one or more categorizations of the patient based on the collected demographic and medical data, established patient care plan guidelines, and goals to be achieved by the patient care plan. Thus, for example, a patient's demographic information and electronic medical records may indicate that the patient is a 40 year old female that has been diagnosed with diabetes. Various pre-established categories and sub-categories may be defined for different types of patients in an ontology based on the various demographic and medical history characteristics, e.g., a category for diabetes patients, a sub-category of patients in the age range of 40 to 50 years old, a sub-sub-category of female patients, and so on.

Similarly, treatment guidelines may be established for defining ways in which to treat various medical maladies with these treatment guidelines having various triggering patient characteristics. For example, a treatment guideline may specify that for female diabetes patients that are in the age range of 40 to 60 years old, the patient should follow a low sugar diet and have at least 30 minutes of stressful exercise per day. A database of such treatments and their guidelines may be provided that correlates various combinations of patient characteristics with a corresponding treatment. Thus, by categorizing the patient in accordance with their characteristic information as obtained from demographic and medical data for the patient, these categories may be used to evaluate the applicability of the various treatments by matching the categories with the patient characteristics of the treatments to identify the best treatment for the patient, i.e. the treatment having the most matches between the patient categories and the treatment's required patient characteristics.

At this point, a general patient care plan is generated for each patient that identifies the treatment, which may be an on-going treatment, which should be prescribed for the patient. A patient care plan in this context is essentially a set of goals and actions for achieving those goals. As will be described hereafter, in addition, the present invention includes, in a patient care plan, a patient monitoring plan with specific actions to be taken on the part of an assessor to monitor and interface with the patient to elicit positive results from the patient, e.g., adherence to the patient care plan.

While a general patient care plan is present at this point, the general patient care plan has not yet been personalized or customized to the specific patient's unique lifestyle information. That is, while in general a 40 year old female diabetes patient should follow a low sugar diet with 30 minutes of stressful exercise each day, not every patient's lifestyle will accommodate such actions in the same way.

The illustrative embodiments further operate to personalize the general patient care plan to the particular lifestyle of each specific patient. Lifestyle information data is obtained from various sources to obtain an overall representation of the lifestyle of the patient. Examples of such sources include geospatial information sources, weather information sources, commercial establishment websites or computing devices/databases, governmental or regulatory organization information sources, and the like. These third-party lifestyle information sources may provide lifestyle information that is combined with lifestyle information provided by the patient himself/herself for analysis to identify the types of personalized care plan actions to be used with the patient's care plan, the timing of the actions, and the types and timing of patient care plan monitoring and management actions to be performed by an assessor, e.g., a human assessor, automated assessment system, or a combination of human and automated assessment mechanisms. Thus, the selection of patient care plan actions (i.e. patient actions and monitoring actions) is based on the general patient care plan goals, the general patient care plan actions to be performed, and the personalization of these general patient care plan actions to the specific lifestyle of the patient.

Various lifestyle information analysis logic is provided to evaluate and classify the patient's lifestyle in accordance with a number of defined lifestyle categories. For example, the patient's lifestyle may be categorized according to level of physical activity, level of availability to healthy food sources, quality of home and work environment (lighting, air quality, quietness, safety, etc.), and level of access to exercise facilities, various qualitative aspects of the patient's home and work life, and the like. From these categories, a more specific patient care plan is generated to achieve the goals and actions of the generic patient care plan, e.g., prescribe a specific type of diet plan which the patient has access to foods that meet with the diet plan and have a schedule that facilitates preparation of particular types of food.

For example, if the patient has limited time due to long work hours, having young children that require attention in the mornings/evenings before/after work, and the like, then food preparation time will be determined to be a minimum and thus, a corresponding diet plan will be selected for this particular type of lifestyle involving more processed foods than another patient that may have more time to perform more complex food preparation actions. Similarly, based on the patient's lifestyle information as obtained from the various sources, the mechanisms of the illustrative embodiments may prescribe a walking regimen based on the fact that the patient lives near a walking trail (as obtained from GPS data) and works in a building that has multiple floors (as obtained from patient supplied lifestyle information, GPS data, and/or governmental real estate databases) such that walking the stairs is an option. The patient's lifestyle information may further indicate an ability to prescribe a strength-building regimen since the patient lives near a gym (obtained from GPS data) or has gym facilities at their office (obtained from the patient supplied lifestyle information and/or real estate database information listing amenities of the building where the patient works). The timing of such actions may be specified in the patient care plan such that the walking regimen may instruct the patient to take a 25 minute walk at 8 a.m. every weekday and walk up/down the stairs at their office on their way to and from work and to and from lunch. The patient care plan may further specify that the patient is to go to the gym on Tuesday and Thursday at 7:30 p.m. to do 30 minutes of strength building exercise.

The granularity of the patient care plan may be even more specific depending upon the implementation. For example, with regard to a walking regimen, a particular path for the patient to walk may be specified in order to achieve a desired level of stress on the patient may be specified based on the geospatial information for the patient's home, work, and other locations, e.g., “Walk up Main Street to 2^{nd} Street, take a left, walk along 2^{nd} Street to Picard Street, take a left, walk down Picard Street to 1^{st} Street, take a left, and return to building.” Such a path determination may be made based on information obtained about the geographical location of the patient's office building including the elevations of the streets to indicate uphill or downhill walking, distances, etc.

Because the lifestyle information may comprise specific establishment information, the patient care plan actions may be further personalized to the patient's particular locations and may specify particular establishments that can be frequented as well as what products/services the patient can utilize to be in compliance with the patient's prescribed care plan. For example, the menu items at a local restaurant may be analyzed to identify which menu items meet the diet requirements of the patient's care plan, e.g., low sugar foods, and the restaurant and its compliant menu items may be provided to the patient as part of their patient care plan. Personal trainer information for gyms may be obtained which includes the personal trainers' schedules, class schedules, and times of availability such that the patient may be instructed, as part of their personal patient care plan, when would be the best time for them to go to the gym to obtain personal trainer assistance with their strength building exercise regimen.

This more PPCP may further be customized to the specific lifestyle of the patient by evaluating the temporal lifestyle information and behavioral lifestyle information for the patient. Thus, having established a set of goals and actions to achieve those goals that are specific to the patient based on their demographics, medical data, and the patient's lifestyle information, the goals and actions may be converted to specific actions to be taken by the patient on a daily basis. For example, the patient's lifestyle information may be further analyzed to identify specific exercise actions to be taken by the patient based on their location, the facilities available, the patient's personal schedule of activities during the day, the patient's personal likes/dislikes (preferences), etc. For example, the patient may have a schedule that shows that the patient is available to exercise between 8 and 9 a.m. and 7:00 p.m. till 8:00 p.m. on most weekdays, is not available Thursday evenings after work for exercise, and is available between 1 and 2 p.m. on Saturdays, and all day on Sundays. The preferences may further state that the patient does not like hot or rainy weather. The patient lifestyle information may further indicate that the patient likes to sleep late on Saturdays and Sundays and thus, while available early on these days, the mechanisms of the illustrative embodiments may adjust the scheduling of actions in the personalized care plan to accommodate this timing preference of the patient. Furthermore, the patient care plan may be dynamically adjusted based on determine weather and temperature conditions, e.g., instead of a standard walking regime that may have been previously part of the patient care plan, because the weather outside indicates a temperature of approximately 90 degrees and 20% chance of rain, the patient care plan may be adjusted to walking for 25 minutes in a neighborhood shopping mall.

It can be appreciated that because the lifestyle information that may be utilized to provide personalization of patient care plans is varied and vast, the types of personalization that may be made to a patient care plan are likewise varied and vast. The patient care plan personalization mechanism of the illustrative embodiments provides logic for analyzing and evaluating a large set of lifestyle information data from various sources, determine specific patient care plan actions that meet the categorization and characterization of the patient's lifestyle as obtained from the analysis of the patient's lifestyle information, as well as achieves the goals and general actions associated with the generalized patient care plan corresponding to the patient's demographics and medical data, and compose the various PPCP actions into a series of actions to be taken by the patient over a set time period, e.g., daily, weekly, monthly, etc., in order to achieve desired goals of the patient care plan.

Having generated a PPCP for a plurality of patients taking into account each patient's personal lifestyle, the illustrative embodiments further provide an engagement optimization engine for generating and implementing a personalized patient engagement plan (PPEP) to monitor each patient's adherence to the PPCP as well as assist health professionals, assessors, automated assessment systems, and the like, in performing actions and initiating communications to maintain ongoing treatment and care of the patient. However, when generating and implementing a PPEP for a plurality of patients, not only do patient-specific factors have to be considered but program-specific factors also have to be considered. That is, while individual PPEPs are relatively simple to generate, the coordination of each of the individual PPEPs for each patient with regard to the resources of the medical facility performing the engagement with each patient also has to be considered. In accordance with the illustrative embodiments, may include but are not limited to, a hospital, doctor's office, emergency care facility, virtual nursing center, telemedicine, telecare, or the like, without departing from the spirit and scope of the invention. That is, if a set of engagement plans for 100 patients require following-up with each a patient though a live engagement with a nurse, then the medical facility is required to have enough nursing resources to make each of the live engagements.

Thus, the mechanisms of the illustrative embodiments take into consideration not only the patient-specific factors of each of the patients but also the program-specific factors of the medical facility. In the illustrative embodiments, the medical facility may be a doctor's office, an emergency healthcare facility, a hospital, or the like. In order to determine an optimal PPEP for engaging with each patient based on the cohort associated with the patient and patient-specific factors, as well as program-specific factors for programs which may be applicable to the patient, the illustrative embodiments provide an engagement optimization engine may then be utilized to output the PPEP and/or implement the PPEP so as to engage with the patient and thereby improve healthcare provided to the patient and reduce subsequent costs of care.

Thus, the engagement optimization engine receives, as an input, a cohort that comprises patients having different levels of risk and different personal characteristics and determines how to assist a medical facility in engaging with each individual patient of the cohort based on patient-specific factors and program-specific factors. For example, a cohort may have multiple patients with high blood pressure, where some patients in the cohort have severe risk of death due to their various personal medical condition factors, while other patients in the cohort may have high blood pressure but have only recently been diagnosed and, as such have significantly lower risk of death. While these patients may all be present in the same cohort, the engagement optimization engine determines how to engage each patient based on their own patient-specific factors and based on the program-specific factors associated with the programs offered by the medical facility for which engagement with the patient is being conducted.

Accordingly, such mechanisms may involve evaluating patient-specific factors including, but not limited to, the medical condition of the patient, a predicted risk level associated with the patient's medical condition (e.g. high risk, low risk, or the like, as determined by risk levels identified as being associated with the particular medical condition in one or more medical knowledge bases), care gaps for the patient (e.g. required treatments, lab tests, or the like), socio-demographics associated with the patient (e.g. age, race, gender, etc. as well as a location of the patient's home, a location of the patient's work, a location of the patient's child's school), opt-ins and preferences indicated by the patient (e.g. email, text, phone calls, or the like, as well as times of day that are best for the patient), engagement history (e.g. how the patient has been engaged in the past and results of that engagement), or the like.

In addition to evaluating the patient-specific factors, the mechanisms also evaluate program-specific factors including, but not limited to, available programs for the patient, delivery costs for delivering the particular types of engagements (e.g., human based engagements, such as by a nurse, are more costly than automated messaging), expected engagement level (e.g., likelihood of responses from the patients), value of the expected outcome from the engagement (e.g., how much does the engagement lower the care costs for the patient), and current capacity of the engagement (e.g., if half of the nurses are out on vacation may not want to recommend human based engagement).

Once the engagement optimization engine identifies the patient-specific factors for each patient in the cohort and the program-specific factors associated with the programs offered by the medical facility for which engagement with each patient is being conducted, the engagement optimization engine performs an optimization such that a PPEP is generated for each patient that meets the needs of the patient (taking into consideration the patient-specific factors) and the programs offered by the medical facility (taking into consideration the program-specific factors) thereby generating a set of PPEPs for each patient in the cohort that optimizes the selection of engagement methodologies for engaging with a particular patient.

Specifically, the engagement optimization engine matches a particular monitoring program to be employed to the specific PPCP that is associated with the patient. That is, for each patient care plan, there may be a set of one or more possible monitoring programs that may be associated with that type of patient care plan. Selection from amongst the one or more possible monitoring program may be performed based on an analysis of the patient's patient-specific factors to determine the most appropriate monitoring program that will not interfere with the patient's patient-specific factors and will most likely result in a positive engagement with the patient. For example, if it is determined that the patient's patient-specific factors is such that the patient leaves for work at 7:30 a.m., then a monitoring program may be selected that involves texting the patient with a message at 7:25 a.m. Other options may be to call the patient or send an electronic mail message but the patient's lifestyle information indicates that the patient is not a “morning person” and thus, is unlikely to respond well to calls in the morning and is generally in a rush to go to work since the patient leaves for work at 8:30 a.m. and needs to be at the office by 9:30 a.m. indicating little time for checking electronic mail.

As with the PPCP, the PPEP and its timing may be personalized to the PPCP and the patient's patient-specific factors. For example, if the patient works in a manufacturing environment where noise levels are high, it is unlikely that the patient will want to conduct a telephone conversation with a human assessor and is more likely to be responsive to textual communications. Thus, during working hours, monitoring programs may be restricted to textual communications, such as instant messaging or electronic mail. Similarly, if the patient works in a hospital, school, or other location where disturbances are to be minimized, communications may not be made during times of the day where the patient is likely to be present in such locations.

Thus, PPEPs are selected based on the patient's patient-specific factors and the program-specific factors of the medical facility. It should be appreciated that as the patient care plan changes over time, the PPEP also changes to match the changes to the patient care plan. Hence, in embodiments where the patient's PPCP is dynamically modified, such as in the case of dynamic changes based on weather, temperature, availability of facilities or resources, etc., the PPEP may likewise be dynamically modified.

In an even further aspect of the illustrative embodiments, the generation of the PPEP, and thus, the monitoring actions of an assessor, may further take into consideration historical analysis of the present patient and other patients in the cohort with regard to the patient's engagement history as well as the engagement history of the other patients in the cohort, e.g. the patients success/failure at responding to the PPEP. That is, historical analysis of patient information is performed across multiple patients to determine which PPEPs resulted in successful engagement with each patient and which resulted in unsuccessful engagement with each patient. Utilizing this information, the engagement optimization engine may change one or more PPEPs for one or more patients in the cohort of patients. For example, if one PPEP indicates that a nurse needed to contact the patient at 8:25 a.m., but 4 out of the 5 time the nurse attempted a contact at 8:25 a.m. the patient failed to respond bet did respond at 12:30 p.m. and another patient has requested that their PPEP be changed from 12:30 p.m. to any time before 9:00 a.m., then engagement optimization engine may modify both of the PPEPs for the patients since there is no additional task requirements for the nurse. This will result in PPEPs for both the patients and the nurse that are tailored to the particular patients, as mentioned above, but in which previous success of other similar patients is taken into account when generating the PPCPs and the PPEPs. This historical analysis can be performed in the aggregate over a plurality of patients and/or on an individual basis based on what this particular patient has shown success, or lack thereof, with in the past.

It should be appreciated that this historical evaluation may be performed at any point during the process of personalizing a patient care plan and PPEP as previously described above. Thus, for example, in one illustrative embodiment, the historical analysis may be performed when generating the generalized patient care plan so as to identify corresponding general patient engagement plan that previously have been most likely achieved by the current and other patients in the cohort. In addition, either in the same or other illustrative embodiments, the historical analysis may be performed when personalizing the generic patient care plans and general patient engagement plans based on the patient's patient-specific factors. That is, historical analysis may be performed based on the patient's previous PPCPs to determine what types of engagements the patient has previously been able to adhere to, which they have not been able to adhere to, or the like.

In yet a further aspect of the illustrative embodiments, mechanisms are provided for dynamically adjusting or modifying PPCPs and thus PPEPs based on a determined level of adherence to the PPCP, as determined from the monitoring actions performed and discussed above during the engagements of the PPEPs. That is, the patient's adherence to their PPCP is monitored during the engagements with the medical professionals and determinations are made as to whether the patient meets the goals set forth in the PPCP and/or performs the patient actions in the PPCP. If the patient does not meet the requirements of one or more goals in the patient care plan, alternative goal determination logic is employed to determine an alternative goal that the patient is more likely to be able to accomplish. This determination may be made based on the patient's actual progress towards attaining the original goal, the importance and type of the goal to the overall PPCP, e.g., adjustments to medication may not be able to be made depending on the particular care plan, and a pre-determined inter-changeability of the goals. In some cases, one goal may be adjusted in one direction, or by a first adjustment metric, and another in a different direction, or by a second adjustment metric, so as to balance the patient's ability to achieve a missed goal with an alternative goal while maintaining overall results that are to be generated, e.g., physical activity goal may be reduced while dietary goals may be increased so that the balance achieves the same overall effect. In this way, the patient's PPCP is further optimized for the particular patient based on the achievability of the goals for that particular patient.

In addition to finding alternative goals for a PPCP, alternative patient actions, and thus corresponding monitoring actions requiring alternative PPEPs, may be identified for patient actions in the patient care plan that the patient has not been able to adhere to. In some illustrative embodiments, the determination of alternative care plan actions for performing the alternative goals may be based on a historical analysis of patient actions in other patient care plans that the patient and/or similar patients have undergone. This historical analysis may identify other similar patient actions that achieved similar results to the patient actions that the patient is found to not be able to achieve in the patient's current PPCP.

Thus, in general, as can be seen from the above description and examples, the mechanisms of the illustrative embodiments combine information about a patient's medical condition, medical history, lifestyle information, geographical location(s), facilities located in these geographical locations(s), products and services available in these geographical location(s), desired goals of the care plan, and other lifestyle information, and personalizes the patient care plan to the patient's particular medical condition, particular lifestyle, and available facilities and resources to provide a specific PPCP for this specific patient that is not widely applicable to generalized categories of patients.

This information may further be used to personalize the assessment activities to be performed by the assessment system/personnel and influence the timing, communication modes, and monitoring actions performed. That is, based on the patient-specific factors and the program-specific factors to monitor the care of the patient as part of the patient's care plan, these goals/actions may be paired with monitoring actions to be taken by an assessor, e.g., a medical professional, other individual whose duty it is to monitor and interface with patients to ensure that they are following a prescribed care plan, or automated system. The monitoring actions may likewise be personalized based on the patient's lifestyle information, geographical information, available products and services in the patient's geographical area(s) of interest (e.g., home, work, etc.), and the like. The assessment tasks may be automatically or semi-automatically performed so as to gather information for monitoring the patient's adherence to the PPCP and either automatically or semi-automatically adjust the PPCP accordingly, send notifications to the patient, notify the doctor, or perform some other desired actions for maximizing the probability that the patient will maintain adherence to the PPCP all the while meeting the needs of the patient in accordance with the capabilities of the accessor.

It should be appreciated that the PPCPs and the PPEP (patient actions performed by the patient and monitoring actions performed by the assessor), may be dynamically adjusted based on the patient's current environmental conditions, changes in schedule, determined deviations from the care plan, and other dynamic conditions that may interfere or otherwise require modification, either temporarily or permanently, of the patient's PPCP. As noted above, such factors as weather conditions, temperature conditions, resource availability (e.g., gym is closed), and the like may require temporary modifications to a patient's PPCP. Other factors, such as the patient moving to a new location, obtaining a new place of employment, or the like, may require more permanent modifications to the patient's PPCP. Such factors may be identified and corresponding modifications initiated taking into account the new temporary/permanent lifestyle changes of the patient.

As shown in the figures, and described hereafter, one or more computing devices comprising a distributed data processing system, may be specifically configured to implement a PPCP system in accordance with one or more of the illustrative embodiments. The configuring of the computing device(s) may comprise the providing of application specific hardware, firmware, or the like to facilitate the performance of the operations and generation of the outputs described herein with regard to the illustrative embodiments. The configuring of the computing device(s) may also, or alternatively, comprise the providing of software applications stored in one or more storage devices and loaded into memory of a computing device for causing one or more hardware processors of the computing device to execute the software applications that configure the processors to perform the operations and generate the outputs described herein with regard to the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of one or more of the illustrative embodiments and is not a general purpose computing device. Moreover, as described hereafter, the implementation of the mechanisms of the illustrative embodiments improves the functionality of the computing device(s) and provides a useful and concrete result that facilitates creation, monitoring, and adjusting PPCPs based on personalized lifestyle information and assessment of patient adherence to the PPCP.

As mentioned above, the mechanisms of the illustrative embodiments may be implemented in many different types of data processing systems, both stand-alone and distributed. Some illustrative embodiments implement the mechanisms described herein in a cloud computing environment. It should be understood in advance that although a detailed description on cloud computing is included herein, implementation of the teachings recited herein are not limited to a cloud computing environment. Rather, embodiments of the present invention are capable of being implemented in conjunction with any other type of computing environment now known or later developed. For convenience, the Detailed Description includes the following definitions which have been derived from the “Draft NIST Working Definition of Cloud Computing” by Peter Mell and Tim Grance, dated Oct. 7, 2009.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models. Characteristics of a cloud model are as follows:
On-demand self-service: a cloud consumer can unilaterally provision computing capabilities, such as server time and network storage, as needed automatically without requiring human interaction with the service's provider.
Broad network access: capabilities are available over a network and accessed through standard mechanisms that promote use by heterogeneous thin or thick client platforms (e.g., mobile phones, laptops, and smart devices).
Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).
Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.
Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service models of a cloud model are as follows:
Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.
Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.
Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems; storage, deployed applications, and possibly limited control of select networking components (e.g., host firewalls).

Deployment models of a cloud model are as follows:
Private cloud: the cloud infrastructure is operated solely for an organization. It may be managed by the organization or a third party and may exist on-premises or off-premises.
Community cloud: the cloud infrastructure is shared by several organizations and supports a specific community that has shared concerns (e.g., mission, security requirements, policy, and compliance considerations). It may be managed by the organizations or a third party and may exist on-premises or off-premises.
Public cloud: the cloud infrastructure is made available to the general public or a large industry group and is owned by an organization selling cloud services.
Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes. A node in a cloud computing network is a computing device, including, but not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. A cloud computing node is capable of being implemented and/or performing any of the functionality set forth hereinabove.

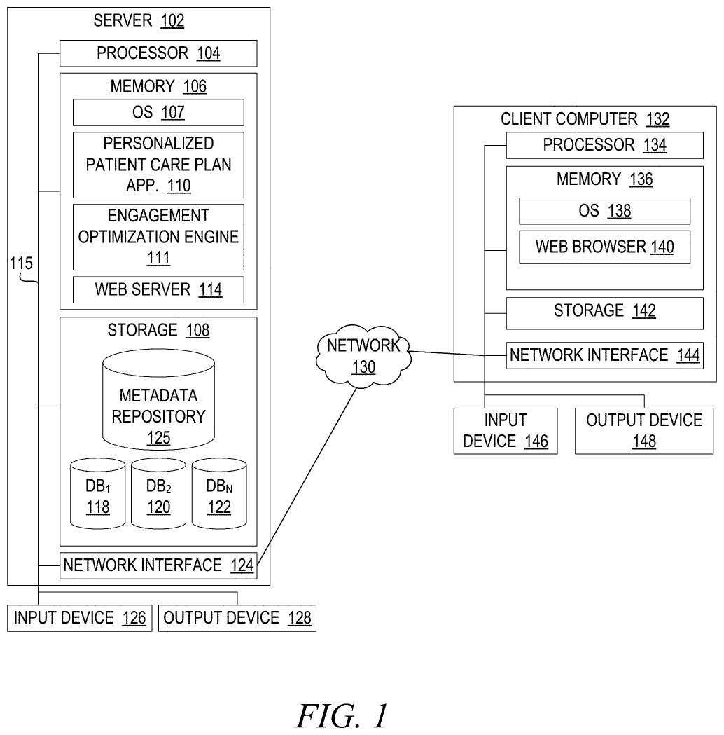

FIG. 1 is a block diagram illustrating a cloud computing system 100 for providing software as a service, where a server provides applications and stores data for multiple clients in databases according to one example embodiment of the invention. The networked system 100 includes a server 102 and a client computer 132. The server 102 and client 132 are connected to each other via a network 130, and may be connected to other computers via the network 130. In general, the network 130 may be a telecommunications network and/or a wide area network (WAN). In a particular embodiment, the network 130 is the Internet.

The server 102 generally includes a processor 104 connected via a bus 115 to a memory 106, a network interface device 124, a storage 108, an input device 126, and an output device 128. The server 102 is generally under the control of an operating system 107. Examples of operating systems include UNIX, versions of the Microsoft Windows™ operating system, and distributions of the Linux™ operating system. More generally, any operating system supporting the functions disclosed herein may be used. The processor 104 is included to be representative of a single CPU, multiple CPUs, a single CPU having multiple processing cores, and the like. Similarly, the memory 106 may be a random access memory. While the memory 106 is shown as a single identity, it should be understood that the memory 106 may comprise a plurality of modules, and that the memory 106 may exist at multiple levels, from high speed registers and caches to lower speed but larger DRAM chips. The network interface device 124 may be any type of network communications device allowing the server 102 to communicate with other computers via the network 130.

The storage 108 may be a persistent storage device. Although the storage 108 is shown as a single unit, the storage 108 may be a combination of fixed and/or removable storage devices, such as fixed disc drives, solid state drives, floppy disc drives, tape drives, removable memory cards or optical storage. The memory 106 and the storage 108 may be part of one virtual address space spanning multiple primary and secondary storage devices.

As shown, the storage 108 of the server contains a plurality of databases. In this particular drawing, four databases are shown, although any number of databases may be stored in the storage 108 of server 102. Storage 108 is shown as containing databases numbered 118, 120, and 122, each corresponding to different types of patient related data, e.g., electronic medical records (EMRs) and demographic information, lifestyle information, treatment guidelines, PPCPs, and the like, for facilitating the operations of the illustrative embodiments with regard to PPCP creation, engagement, monitoring, and modification. Storage 108 is also shown containing metadata repository 125, which stores identification information, pointers, system policies, and any other relevant information that describes the data stored in the various databases and facilitates processing and accessing the databases.

The input device 126 may be any device for providing input to the server 102. For example, a keyboard and/or a mouse may be used. The output device 128 may be any device for providing output to a user of the server 102. For example, the output device 108 may be any conventional display screen or set of speakers. Although shown separately from the input device 126, the output device 128 and input device 126 may be combined. For example, a display screen with an integrated touch-screen may be used.

As shown, the memory 106 of the server 102 includes a personalized patient care plan (PPCP) application 110 with engagement optimization engine 111 configured to provide a plurality of services to users via the network 130. As shown, the memory 106 of server 102 also contains a database management system (DBMS) 112 configured to manage a plurality of databases contained in the storage 108 of the server 102. The memory 106 of server 102 also contains a web server 114, which performs traditional web service functions, and may also provide application server functions (e.g. a J2EE application server) as runtime environments for different applications, such as the patient care plan application 110 and engagement optimization engine 111.

As shown, client computer 132 contains a processor 134, memory 136, operating system 138, storage 142, network interface 144, input device 146, and output device 148, according to an embodiment of the invention. The description and functionality of these components is the same as the equivalent components described in reference to server 102. As shown, the memory 136 of client computer 132 also contains web browser 140, which is used to access services provided by server 102 in some embodiments.

The particular description in FIG. 1 is for illustrative purposes only and it should be understood that the invention is not limited to specific described embodiments, and any combination is contemplated to implement and practice the invention. Although FIG. 1 depicts a single server 102, embodiments of the invention contemplate any number of servers for providing the services and functionality described herein. Furthermore, although depicted together in server 102 in FIG. 1, the services and functions of the PPCP application 110 and engagement optimization engine 111 may be housed in separate physical servers, or separate virtual servers within the same server. The patient care plan application 110 and patient engagement optimization engine 111, in some embodiments, may be deployed in multiple instances in a computing cluster. As is known to those of ordinary skill in the art, the modules performing their respective functions for the PPCP application 110 and engagement optimization engine 111 may be housed in the same server, on different servers, or any combination thereof. The items in storage, such as metadata repository 125, databases 118, 120, and 122, may also be stored in the same server, on different servers, or in any combination thereof, and may also reside on the same or different servers as the application modules.

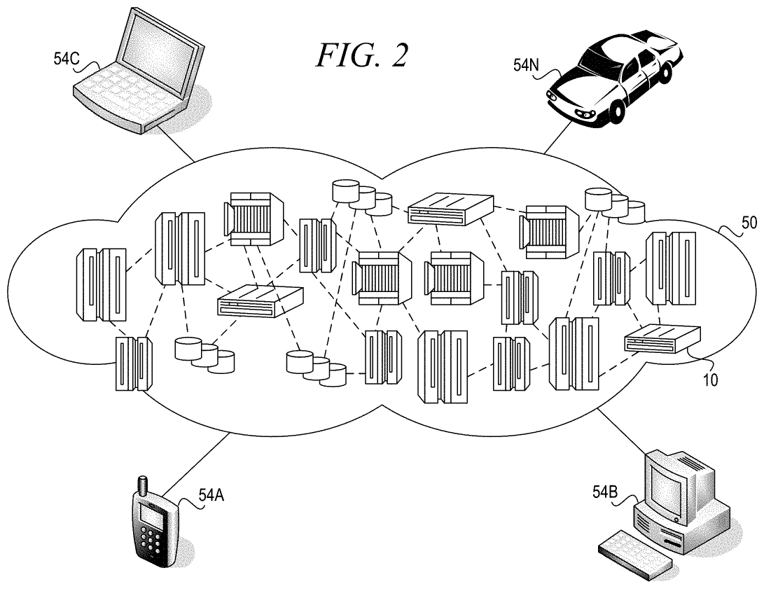

Referring now to FIG. 2, another perspective of an illustrative cloud computing environment 250 is depicted. As shown, cloud computing environment 250 comprises one or more cloud computing nodes 210, which may include servers such as server 102 in FIG. 1, with which local computing devices used by cloud consumers, such as, for example, smart device or cellular telephone 254A, desktop computer 254B, laptop computer 254D, and/or automobile computer system 254N may communicate. Nodes 210 may communicate with one another. A computing node 210 may have the same attributes as server 102 and client computer 132, each of which may be computing nodes 210 in a cloud computing environment. They may be grouped (not shown) physically or virtually, in one or more networks, such as Private, Community, Public, or Hybrid clouds as described hereinabove, or a combination thereof. This allows cloud computing environment 250 to offer infrastructure, platforms and/or software as services for which a cloud consumer does not need to maintain resources on a local computing device. It is understood that the types of computing devices 254A-N shown in FIG. 2 are intended to be illustrative only and that computing nodes 210 and cloud computing environment 250 can communicate with any type of computerized device over any type of network and/or network addressable connection (e.g., using a web browser).

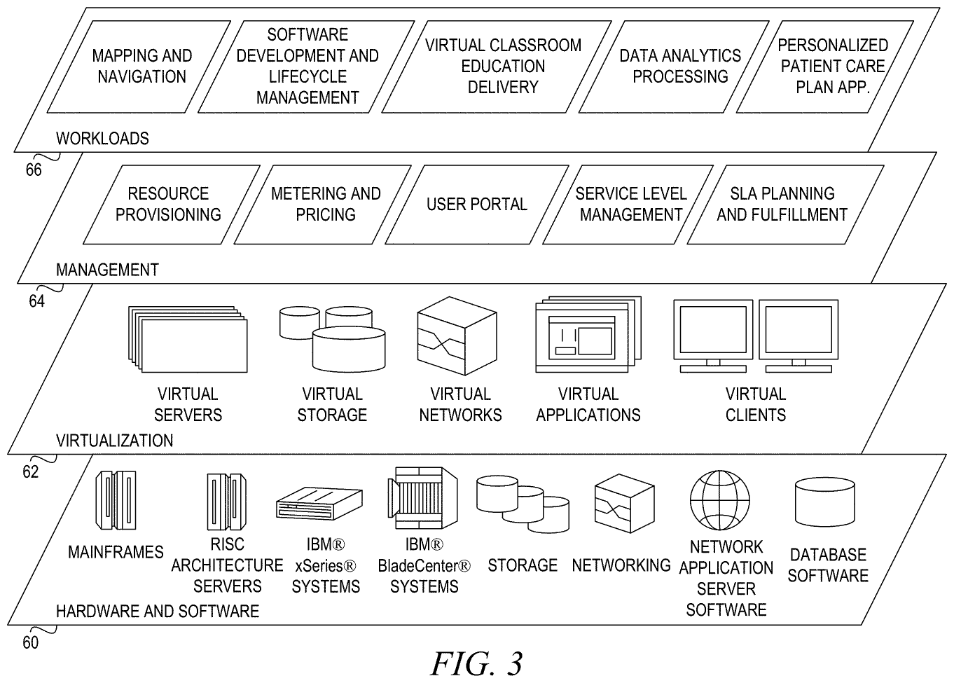

Referring now to FIG. 3, a set of functional abstraction layers provided by cloud computing environment 250 (FIG. 2) is shown. It should be understood in advance that the components, layers, and functions shown in FIG. 3 are intended to be illustrative only and embodiments of the invention are not limited thereto. As depicted, the following layers and corresponding functions are provided.

The hardware and software layer 360 includes hardware and software components. Examples of hardware components include mainframes, in one example IBM™ zSeries™ systems; RISC (Reduced Instruction Set Computer) architecture based servers, in one example IBM pSeries™ systems; IBM xSeries™ systems; IBM BladeCenter™ systems; storage devices; networks and networking components. Examples of software components include network application server software, in one example IBM WebSpherer™ application server software; and database software, in one example IBM DB2™ database software. (IBM, zSeries, pSeries, xSeries, BladeCenter, WebSphere, and DB2 are trademarks of International Business Machines Corporation registered in many jurisdictions worldwide).

The virtualization layer 362 provides an abstraction layer from which the following examples of virtual entities may be provided: virtual servers; virtual storage; virtual networks, including virtual private networks; virtual applications and operating systems; and virtual clients. In one example, management layer 364 may provide the functions described below. Resource provisioning provides dynamic procurement of computing resources and other resources that are utilized to perform tasks within the cloud computing environment. Metering and Pricing provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal provides access to the cloud computing environment for consumers and system administrators. Service level management provides cloud computing resource allocation and management such that required service levels are met. Service Level Agreement (SLA) planning and fulfillment provides pre-arrangement for, and procurement of, cloud computing resources for which a future requirement is anticipated in accordance with an SLA.

Workloads layer 366 provides examples of functionality for which the cloud computing environment may be utilized. Examples of workloads and functions which may be provided from this layer include: mapping and navigation; software development and lifecycle management; virtual classroom education delivery; data analytics processing; transaction processing; and, in accordance with the mechanisms of the illustrative embodiments, a PPCP creation and monitoring functionality.

It should be noted that the cloud computing system described in FIGS. 1-3 may be a cognitive system for implementing an engagement optimization engine for generating and implementing a personalized patient engagement plan (PPEP) to monitor each patient's adherence to the PPCP as well as assist health professionals, assessors, automated assessment systems, and the like, in performing actions and initiating communications to maintain ongoing treatment and care of the patient. Such cognitive systems may improve medical practitioners' treatment of patients and can assist patient in obtaining the specific treatment for their particular medical malady. It should be appreciated that the cognitive system may have multiple request processing pipelines, each request processing pipeline being separately trained and/or configured to process requests associated with different domains or be configured to perform the same or different analysis on input requests, depending on the desired implementation. For example, in some cases, a first request processing pipeline may be trained to operate on input requests directed to an engagement optimization engine to generate and implement a PPEP to monitor each patient's adherence to the PPCP.

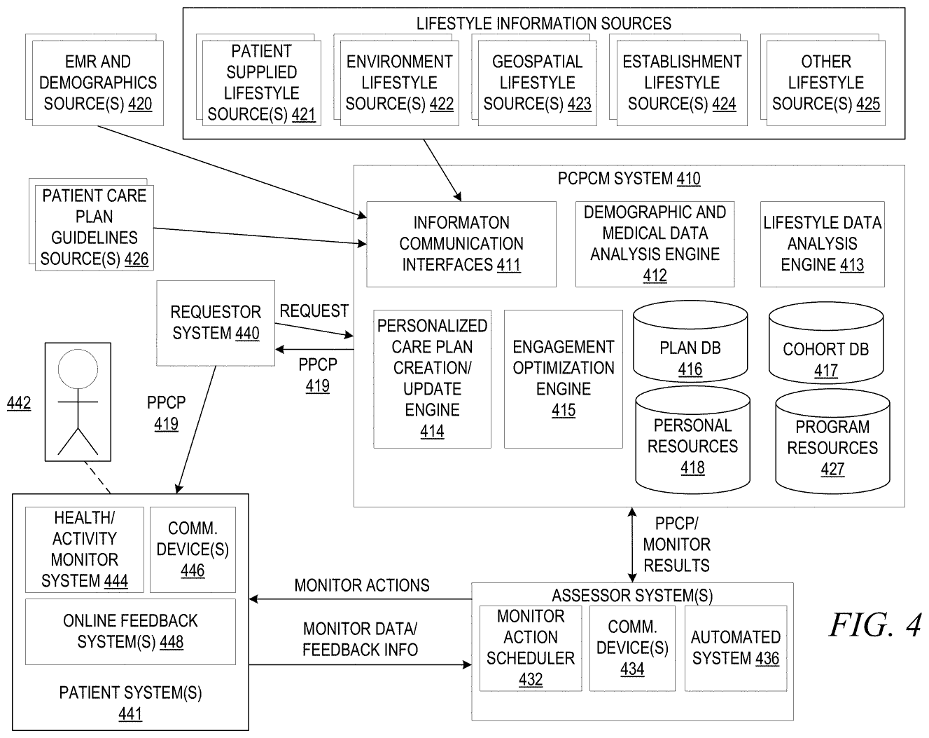

As discussed above, the illustrative embodiments provide a PPCP creation and monitoring system which may be implemented in various types of data processing systems. FIG. 4 is an example block diagram illustrating the primary operational elements of such a personalized patient care plan creation and monitoring system in accordance with one illustrative embodiment. The operational elements shown in FIG. 4 may be implemented as specialized hardware elements, software executing on hardware elements, or any combination of specialized hardware elements and software executing on hardware elements without departing from the spirit and scope of the present invention.

As shown in FIG. 4, a personalized patient care plan creation and monitoring (PCPCM) system 410 comprises information source interfaces 411, demographic and medical data analysis engine 412, lifestyle data analysis engine 413, personalized care plan creation/update engine 414, and engagement optimization engine 415. In addition, the PCPCM system 410 maintains a personalized patient care plan (PPCP) database 416 that stores data corresponding to the PPCPs generated for various patients and a patient cohort database 417 that stores cohort association information for various patients having similar characteristics, e.g., demographics and/or medical data. Entries in the PPCP database 416 may be associated with entries in the patient cohort database 417.

Personalization resources 418 provides resources utilized by the personalized care plan creation/update engine 414 for identify and correlating demographic, medical, lifestyle information, and general patient care plan information associated with a patient into a series of PPCP actions and corresponding PPEPs for the patient and an assessor. The personalization resources 418 may comprise systems of rules, patterns, equations, algorithms, and various other types of logic that codify or otherwise implement functions for selecting and deciding how to personalize a general set of goals and actions in a general patient care plan to a PPCP. These rules, patterns, equations, algorithms, and the like, may be developed over time by subject matter experts. The rules, patterns, equations, algorithms, etc., may be applied to the large set of demographic, medical, and lifestyle information obtained for the patient to obtain an automatically generated PPCP which may then be presented to a subject matter expert, such as a doctor, nurse, other medical professional, or the like, for confirmation before prescribing the PPCP to the patient. It should be appreciated that the personalization resources 418 may further be utilized by the engagement optimization engine 415 when generating and implementing engaging with the patient in order to monitor adherence to a PPCP and determining modifications to the personalize patient care plan based on determined levels of adherence, as discussed hereafter.

The information source interfaces 411 provides a data communication interface through which patient data may be obtained from various sources including electronic medical records (EMRs) data source 420, patient supplied lifestyle data source 421, environment lifestyle information source 422, geospatial lifestyle information source 423, establishment lifestyle information source 424, and other various lifestyle information data sources 425. Moreover, the interfaces 411 comprise interfaces for obtaining patient care plan guidelines information from source 426. The EMR data source 420 may comprise various sources of electronic medical records including individual doctor medical practice systems, hospital computing systems, medical lab computing systems, personal patient devices for monitoring health of the patient, dietary information, and/or activity information of the patient, or any other source of medical data that represents a particular patient's current and historical medical condition. The EMR data source 420 may further comprise data representing the patient demographics since such information is typically gathered by providers of such medical data.

The patient supplied lifestyle data source 421 may be a database and/or computing system that gathers and stores information from the patient indicating the patient's response to questionnaires, presented either physically and then entered through a data entry process or presented electronically and gathered automatically, directed to the patient's lifestyle, preferences, and the like. For example, questions in the questionnaire may ask questions about the patient's personal daily schedule, home and work environment conditions, family information, preferences regarding food types, exercise types, times of the day for performing actions, and the like. This information is gathered directly from the patient but may not cover all aspects of the patient's lifestyle. This lifestyle information may be augmented by other lifestyle information gathered from other sources which may be third-party lifestyle information sources. These third-party lifestyle information may comprise information from commercial and governmental computing systems, databases, and the like, that characterize the patient's environment, availability to resources (e.g., products/services/facilities), etc.

In the depicted example, third-party lifestyle information sources comprise environment lifestyle information source 422, geospatial lifestyle information source 423, establishment lifestyle information source 424, and other various lifestyle information data sources 425. Examples of environment lifestyle information source 422 comprise weather information services, air quality information services, traffic information services, crime information services, governmental information services regarding public utilities, or any other environment lifestyle information source 424. As one example, a third-party geospatial lifestyle information source 423 may comprise a global positioning system (GPS) source that identifies the patient's associated locations, e.g., home, work, etc., and identifies establishments around those locations that provide resources that are of interest to the patient's lifestyle and potentially of interest in generating a patient care plan. For example, as mentioned above, specialty grocery stores, vitamin stores, pharmacies, restaurants, gyms, walking paths, parks, recreational areas, community pools, and the like, may be identified based on a GPS system and its associated databases of information.

The information from the geospatial lifestyle information source 423 may be used to request or lookup establishment information in the establishment lifestyle information source 424. For example, if the geospatial lifestyle information source 423 identifies an establishment type and specific identity of a particular establishment, this information may be used to request or lookup other third-party lifestyle information for the establishment in the establishment lifestyle information source 424, e.g., the establishment's website, an industry based website, blogs, commercial establishment information repository, or the like, to retrieve specific information about the identified establishment, e.g., menu items, nutrition information, hours of operation, and the like. Similarly, other third-party lifestyle information source 425 may provide information for correlation with patient care plan actions/tasks including hours of operations, products/services provided, distance from the patient's locations, and the like.

The patient care plan guidelines source 426 provides information regarding the preferred treatments for various medical conditions or maladies in association with patient characteristics. These guidelines are generally associated with demographic and medical information about patients and provide general guidelines as to who qualifies for a treatment, or patient care plan, and who does not base on their medical information and demographic information. The patient care plan guidelines provide an initial basis for determining a general patient care plan for a patient which may then be personalized to the particular patient based on the lifestyle information specific to that particular patient.

The PCPCM system 410 may receive a request to generate a PPCP for a particular patient, such as from a physician's computing system, a patient computing system, or the like, which initiates the processes of the PCPCM system 410 including retrieving information about the specified patient from the EMR sources 420. The EMR sources 420 provide patient demographic and medical data, gathered from questionnaires, electronic medical records, and the like, to the medical data analysis engine 412 which analyzes the received data and extracts the necessary data for generating patient care plan from the demographic and medical data received. This information is then used as a basis for submitting a request to the patient care plan guidelines source 426 to retrieve patient care plan guidelines for the patient's specific demographics and medical data, e.g., the patient is a 40 year old female diagnosed with type 2 diabetes and thus, corresponding patient care plan guidelines for this combination of patient demographics and medical condition are retrieved from the patient care plan guidelines source 426.

The retrieved patient care plan guidelines are used along with the demographics and medical data for the patient to generate a baseline patient care plan based on an initial diagnosis of the patient's medical condition, one or more categorizations of the patient based on the collected demographic and medical data, the established patient care plan guidelines, and goals to be achieved by the patient care plan, such as may be specified in the established patient care plan guidelines and/or patient medical data. These operations are performed by the PCPCM system 410 utilizing the personalization resources 418 which provide the rules, logic, equations, algorithms and other logic for evaluating patient information and correlating that information with a patient care plan that comprises patient actions to be performed by the patient and monitoring actions to be performed by the assessor. It should be appreciated that based on the demographic information about the patient and the patient's medical data, only a general patient care plan is generated at this point.

The resulting general patient care plan generated by the personalized care plan creation/update engine 414 is then personalized based on the lifestyle information for the patient obtained via the lifestyle data analysis engine 413 convert the general patient care plan to a PPCP for the specific patient based on their own unique combination of lifestyle information. The lifestyle data analysis engine 413 obtains the lifestyle information from the various sources 421-425 and performs analysis to generate lifestyle inferences from the lifestyle data. Again, resources may be provided in the personalization resources 418 for providing logic, algorithms, rules, patterns, etc., for drawing these inferences from the received lifestyle information. For example, from schedule data for the patient, geospatial lifestyle information, environment lifestyle information, and the like for the patient, it may be determined, based on rules, patterns, algorithms, and the like, that the patient has a sedentary occupation, works in a multi-story building that has a gym, lives in an area with access to parks and walking paths, and the like. As one example, the lifestyle information may indicate that the patient's occupation is a lawyer. From that information, a lookup of the occupation in an occupation database provided in the personalization resources 418 may indicate characteristics of the occupation including characteristics of “stressful”, “sedentary”, and “long hours” which provides lifestyle inferences about the patient that can be utilized by rules in the personalization resources 418 implemented by the personalized care plan creation/update engine 414 to personalize the general patient actions in the general patient care plan to the particular patient. Various analysis of lifestyle information may be used to extract such inferences from the data which can then be used to personalize a general patient care plan.

As mentioned above, lifestyle information data is obtained from various sources 421-425 to obtain an overall representation of the lifestyle of the patient. These third-party lifestyle information sources 422-425 may provide lifestyle information that is combined with lifestyle information provided by the patient himself/herself 421 for analysis to identify the types of personalized care plan actions to be used with the patient's care plan, the timing of the actions, and the types and timing of patient care plan monitoring and management actions to be performed by an assessor, e.g., a human assessor, automated assessment system, or a combination of human and automated assessment mechanisms. Thus, the selection of patient care plan actions (i.e. patient actions and monitoring actions) is based on the general patient care plan goals, the general patient care plan actions to be performed, and the personalization of these general patient care plan actions to the specific lifestyle of the patient.

Various lifestyle information analysis logic is provided in the lifestyle data analysis engine 413 to evaluate and classify the patient's lifestyle in accordance with a number of defined lifestyle categories. For example, the patient's lifestyle may be categorized according to level of physical activity, level of availability to healthy food sources, quality of home and work environment (lighting, air quality, quietness, safety, etc.), level of access to exercise facilities, various qualitative aspects of the patient's home and work life, and the like. From these categories, a more specific patient care plan is generated to achieve the goals and actions of the generic patient care plan. Non-limiting examples of ways in which general patient care plans may be personalized based on lifestyle information have been provided above. Such personalization may be performed by the personalized care plan creation/update engine 414.

It should be appreciated that the personalization resources 418 may comprise various reference resources from which the mechanisms of the PCPCM system 410 may obtain information for making decisions as to how to personalize the patient care plan actions (patient actions and monitoring actions). Such reference resources may comprise drug information repositories, food nutrition repositories, exercise information repositories, medical procedure repositories, and the like. The “reference” resources differ from other lifestyle information sources in that these “reference” resources tend to be universal for all patients. Such reference resources may be utilized, for example, to assist in determining drug effects on other lifestyle characteristics (e.g., drugs that make one lethargic, prone to disorientation, or the like), selecting foods whose nutritional content falls within the desired goals of a patient care plan, selecting exercises that generate a desired level of activity within a given period of time, and the like.

It should be appreciated that in addition to the evaluation of the patient's demographic, medical, and lifestyle information, the personalized care plan creation/update engine 414 may evaluate the historical personalized care plan information for a patient and for other similar patients to determine appropriate patient actions to include in a personalized care plan. For example, the personalized care plan creation/update engine 414 may look to a history of personalized care plans created for this patient, as may be maintained in the PPCP database 416 in association with an identifier of the patient, to determine what patient actions the patient was able to successfully complete in previously prescribed PPCPs and use this information to select those same patient actions for a current PPCP should the current PPCP have similar goals, general patient actions, and the like that the previously successful patient actions would satisfy. Thus, when selecting personalized patient actions to include in the PPCP, different weightings may be applied to patient actions based on whether or not they were previously prescribed to this patient, whether or not they were previously successfully completed by the patient in previously prescribed PPCPs, a level of successful or non-successful completion of the patient action in previously prescribed PPCPs, as well as published clinical best practices, i.e. those care practices that may have changed between a previous care time and a current care time. A highest ranking patient action, amongst the possible patient actions, may then be selected for inclusion in the PPCP.

In addition, the personalized care plan creation/update engine 414 may retrieve information from the patient cohort database 417 to classify the patient into a patient cohort. The patient cohort is a grouping of patients that have similar characteristics, e.g., similar demographics, similar medical diagnoses, etc. Patient cohorts may be generated using any known or later developed grouping mechanism. One example mechanism may be using a clustering algorithm that clusters patients based on key characteristics of the patient, e.g., age, gender, race, medical diagnosis, etc. With regard to the illustrative embodiments, the present patient may be grouped into a patient cohort and the other members of the patient cohort may be evaluated to identify patient actions that the other members were able to successfully complete as part of their PPCPs. These patient actions may then be provided for use in generating the PPCP for the present patient, with appropriate weightings applied to rank these patient actions relative to other patient actions for purposes of selection as discussed above.

Thus, the PCPCM system 410 provides the various mechanisms for providing actual PPCPs based not only on a categorization of the patient based on their medical diagnosis and demographic information, but also based on their own specific lifestyle information and lifestyle information obtained from third-party sources. In addition, the PCPCM system 410 further provides the mechanisms for generating, as part of the PPCP, monitoring actions to be performed by an assessor in monitoring the patient's performance of the patient actions of the PPCP. That is, based on the creation of the series of patient actions to be performed by the patient over a designated period of time, e.g., daily, weekly, monthly, etc., corresponding monitoring actions are identified using the personalization resources 418. The personalization resources 418 may comprise rules, logic, patterns, algorithms, etc. that match monitoring actions to types of patient actions.

Based on timing information for the patient actions, preferences specified by the patient in the patient supplied lifestyle information 421, and the like, engagement optimization engine 415 generates and implements a personalized patient engagement plan (PPEP) 428 to monitor each patient's adherence to the personalized care plan as well as assist health professionals, assessors, automated assessment systems, and the like, in performing actions and initiating communications to maintain ongoing treatment and care of the patient.

Thus, engagement optimization engine 415 takes into consideration not only the patient-specific factors in the lifestyle information from the various sources 421-425 but also the program-specific factors of the medical facility in program resources 427. Again, the patient-specific factors in the lifestyle information from the various sources 421-425 include, but are not limited to, the medical condition of the patient, a predicted risk level associated with the patient's medical condition (e.g. high risk, low risk, or the like, as determined by risk levels identified as being associated with the particular medical condition in one or more medical knowledge bases), care gaps for the patient (e.g. required treatments, lab tests, or the like), socio-demographics associated with the patient (e.g. age, race, gender, etc. as well as a location of the patient's home, a location of the patient's work, a location of the patient's child's school), opt-ins and preferences indicated by the patient (e.g. email, text, phone calls, or the like, as well as times of day that are best for the patient), engagement history (e.g. how the patient has been engaged in the past and results of that engagement), or the like.

The patient-specific factors of the medical facility in program resources 427 includes, but is not limited to, available programs for the patient, delivery costs for delivering the particular types of engagements (e.g., human based engagements, such as by a nurse, are more costly than automated messaging), expected engagement level (e.g., likelihood of responses from the patients), value of the expected outcome from the engagement (e.g., how much does the engagement lower the care costs for the patient), and current capacity of the engagement (e.g., if half of the nurses are out on vacation may not want to recommend human based engagement).

In the illustrative embodiments, the medical facility may be a doctor's office, an emergency healthcare facility, a hospital, or the like. In order to determine an optimal engagement plan for engaging with each patient based on the cohort associated with the patient from the patient cohort database 417 and patient-specific factors, as well as program-specific factors for programs which may be applicable to the patient, the engagement optimization engine 415 receives, as an input, the cohort from the patient cohort database 417 that comprises patients having different levels of risk and different personal characteristics and determines how to assist a medical facility in engaging with each individual patient of the cohort based on patient-specific factors and program-specific factors. While these patients may all be present in the same cohort, the engagement optimization engine 415 determines how to engage each patient based on their own patient-specific factors and based on the program-specific factors associated with the programs offered by the medical facility for which engagement with the patient is being conducted.

Once engagement optimization engine 415 identifies the patient-specific factors for each patient in the cohort and the program-specific factors associated with the programs offered by the medical facility for which engagement with each patient is being conducted, the engagement optimization engine performs an optimization such that PPEP 428 is generated for each patient that meets the needs of the patient (taking into consideration the patient-specific factors) and the programs offered by the medical facility (taking into consideration the program-specific factors) thereby generating a set of PPEPs 428 for each patient in the cohort that optimizes the selection of engagement methodologies for engaging with a particular patient.

Specifically, the engagement optimization engine 415 matches a particular monitoring program from program resources 427 to be employed to the specific PPCP that is associated with the patient. That is, for each patient care plan, there may be a set of one or more possible monitoring programs that may be associated with that type of patient care plan. Selection from amongst the one or more possible monitoring program may be performed based on an analysis of the patient's patient-specific factors to determine the most appropriate monitoring program that will not interfere with the patient's patient-specific factors and will most likely result in a positive engagement with the patient. The PPEP 428 may be scheduled as part of the PPCP monitor, e.g., every day the leaves for work at 7:30 a.m., therefore schedule a monitoring action at 7:25 a.m. to send a text message to the patient's communication device to engage the patient.

Thus, the resulting PPCP 419 and the PPEP 428 comprise patient/assessor engagements requiring actions to be performed by the patient and actions to be performed by the assessor. Having generated a PPCP taking into account the patient's personal lifestyle, the PCPCM system 410 outputs the PPCP 419 to the requestor system 440 for use by the patient 442 in performing the patient actions of the PPCP. In addition, as noted above, the PPEP 428 further comprises how/when the actions are to be monitored by an assessor via assessor systems 430, which may be a human being utilizing communications and/or computing equipment 432-436 to perform their monitoring actions, an automated system 436 that automatically performs monitoring actions, or a combination of human and automated systems. The PPCP 419 and the PPEP 428 are output to the assessor system(s) 430 such that the assessor may utilize the monitoring actions in the PPCP 419 to monitor and evaluate the patient's performance of the patient actions according to the PPEP 428.

In monitoring the patient 442 and the patient's adherence to the PPCP 419, the assessor system(s) 430 may obtain feedback information from various patient systems 441 including a health/activity monitor system 444, communication device(s) 446, online feedback system(s) 448, or the like. Examples of health/activity monitor system 444 include wearable devices, such as a FitBit™, iFit™ Fitness Tracker, pedometers, medical equipment with data connectivity to one or more networks via wired or wireless data communication links, or the like. Examples of communication device(s) 446 may include smart phones with applications for communication via data networks to log health and activity data for the patient 442, conventional phones through which a human or automated mechanism places calls to the patient 442, or the like. Examples of online feedback system(s) 448 include websites for tracking a patient's medical condition including online food logs, weight monitoring services, and other health and activity monitoring systems. Any systems that facilitate monitoring and/or communication with an assessor may be used as part of the patient system(s) 441 without departing from the spirit and scope of the illustrative embodiments.

Examples of monitoring actions performed by the assessor system(s) 430 may include interrogating the health/activity monitoring devices and/or applications executing on the communication devices 446 or online feedback system(s) 448 associated with the patient, and initiating a reminder communication to be sent to the patient's communication device 446 via the assessor communication device 434 to remind the patient 442 to perform an action in accordance with their PPCP 419, scheduling a doctor's appointment for the patient and informing them of the appointment, initiating a call to the patient's communication device 446 to discuss their progress, or any other action that a human or automated assessment system 436 may perform to assist with the monitoring of the patient's adherence to the patients' PPCP 419. Moreover, results of the monitoring may be returned to the PCPCM system 410 for use in modifying the PPCP 419 based on the patient's determined level of adherence to the PPCP 419.

In response to monitoring results and feedback gathered by the assessor system(s) 430, and provided back to the PCPCM system 410, the personalized care plan creation/update engine 414 may dynamically adjust or modify the PPCP 419 based on a determined level of adherence to the PPCP 419. That is, the patient's adherence to their PPCP 419 is monitored via the assessor system(s) 430 and the patient system(s) 441, and determinations are made as to whether the patient meets the goals set forth in the PPCP 419 and/or performs the patient actions in the PPCP 419. If the patient does not meet the requirements of one or more goals in the patient care plan 419, alternative goal determination logic of the personalized care plan creation/update engine 414 is employed to determine an alternative goal that the patient is more likely to be able to accomplish. This determination may be made based on the patient's actual progress towards attaining the original goal, the importance and type of the goal to the overall PPCP, e.g., adjustments to medication may not be able to be made depending on the particular care plan, and a pre-determined inter-changeability of the goals. These determinations may be made in a similar manner as previously described above with regard to the original generation of the PPCP utilizing the personalization resources 418 and the like, with the adherence feedback and monitoring data being used as additional lifestyle information for influencing the selection of patient actions and corresponding monitoring actions.

Once the personalized care plan creation/update engine 414 adjusts or modifies the PPCP 419 based on a determined level of adherence to the original PPCP 419, engagement optimization engine 415 may adjust or modify the PPEP 428 based on the adjusted or modified PPCP 419 and/or the patient's history of access during the engagements associated with the original PPCP 419. Specifically, if the adjusted or modified PPCP 419 requires new or different monitoring actions, then the engagement optimization engine 415 has to adjust or modify the original PPEP accordingly. Additionally or in lieu of, if the patient has been unavailable during previous engagements either with the original PPCP 419 or with a previous personalized care plan, then the engagement optimization engine 415 adjusts or modifies the PPEP 428 based on the historical information.

It should be appreciated that the patient systems may further comprise systems for identifying the current location, environmental conditions, changes in a schedule, and the like, for use by the assessor systems 430 in providing feedback to the PCPCM system 410 to adjust the PPCP 419 and engagement optimization engine 415 to adjust the PPEP 428 for the patient's current location and environment. That is, the PPCP 419 and PPEP 428 may be dynamically adjusted based on the patient's current environmental conditions, changes in schedule, determined deviations from the care plan, and other dynamic conditions that may interfere or otherwise require modification, either temporarily or permanently, of the patient's PPCP and the PPEP. As noted above, such factors as weather conditions, temperature conditions, resource availability (e.g., gym is closed), and the like may require temporary modifications to a patient's PPCP and the PPEP. Other factors, such as the patient moving to a new location, obtaining a new place of employment, or the like, may require more permanent modifications to the patient's PPCP and the PPEP. Such factors may be identified and corresponding modifications initiated taking into account the new temporary/permanent lifestyle changes of the patient.

From the above general overview of the mechanisms of the illustrative embodiments, it is clear that the illustrative embodiments are implemented in a computing system environment and thus, the present invention may be implemented as a data processing system, a method implemented in a data processing system, and/or a computer program product that, when executed by one or more processors of one or more computing devices, causes the processor(s) to perform operations as described herein with regard to one or more of the illustrative embodiments. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

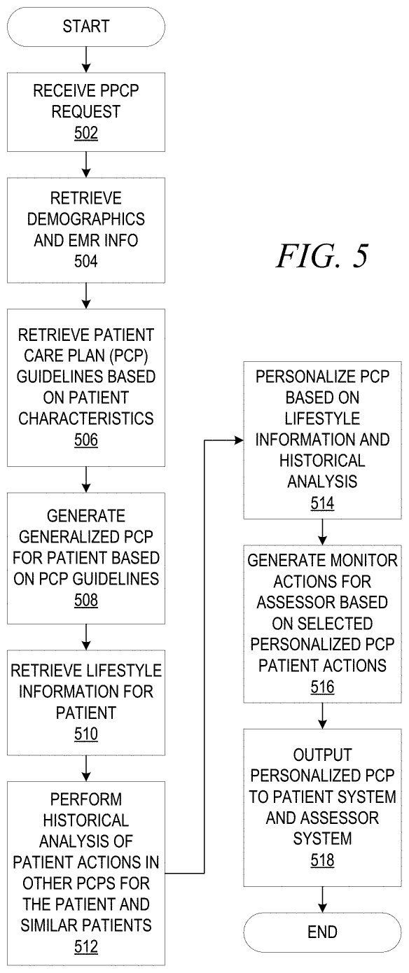

FIG. 5 is a flowchart outlining an example operation for creating a personalized patient care plan in accordance with one illustrative embodiment. As shown in FIG. 5, the operation comprises receiving a request (Personalized Patient Care Plan (PPCP) request) for the creation of a PPCP specifically identifying a patient for which the PPCP is to be created (step 502). EMR and demographic information is retrieved for the patient (step 504) and used to retrieve one or more patient care plan guidelines corresponding to the patient's characteristics (step 506). A generalized patient care plan (PCP) is generated for the patient based on the retrieved PCP guidelines and the patient's demographics and medical information (step 508).

Patient specific lifestyle information is retrieved for the patient from a plurality of different lifestyle information sources (step 510). Moreover, in some illustrative embodiments, a historical analysis is performed on patient actions in previously prescribed PCPs for this patient and similar patients (such as patients in a same cohort) to identify patient actions that are ones that the patient is likely to be able to adhere to and weight them more heavily during a selection process (step 512). A personalized PCP is generated based on the generalized PCP as a basis which is then customized and personalized to the specific patient using the retrieved lifestyle information, the historical analysis results identifying patient actions that are likely to be adhered to by this patient, published clinical best practices, and established rules, patterns, algorithms, logic, etc., for generating personalized patient actions and combining them in a serial manner to generate a sequence of patient actions and goals that together constitute the patient's side of the PPCP (step 514). Based on the selected patient actions in the PPCP, corresponding monitor actions for all or a subset of the patient actions are generated using monitoring action rules, patterns, algorithms, logic, or the like (step 516). The monitoring actions are combined with the patient actions in the personalized PCP (PPCP) which is then output to the patient system(s) and assessor system(s) for implementation and monitoring of the PPCP (step 518). The operation then ends.

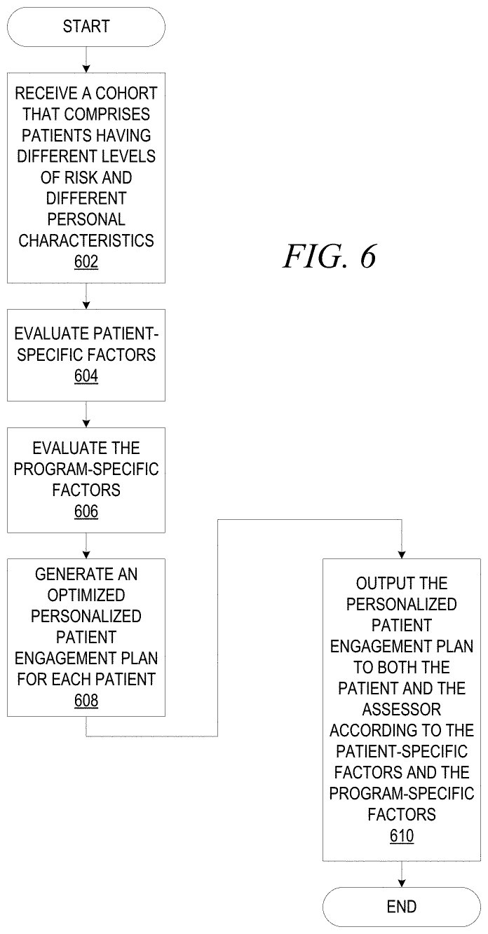

FIG. 6 is a flowchart outlining an example operation for generating a personalized patient engagement plan (PPEP) in accordance with one illustrative embodiment. As shown in FIG. 6, the operation starts by the engagement optimization engine receiving, as an input, a cohort that comprises patients having different levels of risk and different personal characteristics (step 602). For example, a cohort may have multiple patients with high blood pressure, where some patients in the cohort have severe risk of death due to their various personal medical condition factors, while other patients in the cohort may have high blood pressure but have only recently been diagnosed and, as such have significantly lower risk of death. While these patients may all be present in the same cohort, the engagement optimization engine determines how to engage each patient based on their own patient-specific factors and based on the program-specific factors associated with the programs offered by the medical facility for which engagement with the patient is being conducted.

Upon receiving the cohort, the engagement optimization engine evaluates patient-specific factors (step 604) including, but not limited to, the medical condition of the patient, a predicted risk level associated with the patient's medical condition (e.g. high risk, low risk, or the like, as determined by risk levels identified as being associated with the particular medical condition in one or more medical knowledge bases), care gaps for the patient (e.g. required treatments, lab tests, or the like), socio-demographics associated with the patient (e.g. age, race, gender, etc. as well as a location of the patient's home, a location of the patient's work, a location of the patient's child's school), opt-ins and preferences indicated by the patient (e.g. email, text, phone calls, or the like, as well as times of day that are best for the patient), engagement history (e.g. how the patient has been engaged in the past and results of that engagement), or the like.

The engagement optimization engine then evaluates the program-specific factors (step 606) including, but not limited to, available programs for the patient, delivery costs for delivering the particular types of engagements (e.g., human based engagements, such as by a nurse, are more costly than automated messaging), expected engagement level (e.g., likelihood of responses from the patients), value of the expected outcome from the engagement (e.g., how much does the engagement lower the care costs for the patient), and current capacity of the engagement (e.g., if half of the nurses are out on vacation may not want to recommend human based engagement).

Once the engagement optimization engine identifies the patient-specific factors for each patient in the cohort and the program-specific factors associated with the programs offered by the medical facility for which engagement with each patient is being conducted, the engagement optimization engine generates an optimized PPEP for each patient (step 608). Each PPEP meets the needs of the patient (taking into consideration the patient-specific factors) and the programs offered by the medical facility (taking into consideration the program-specific factors) thereby generating a set of PPEPs for each patient in the cohort that optimizes the selection of engagement methodologies for engaging with a particular patient.

Specifically, the engagement optimization engine matches a particular monitoring program to be employed to the specific PPCP that is associated with the patient. That is, for each patient care plan, there may be a set of one or more possible monitoring programs that may be associated with that type of patient care plan. Selection from amongst the one or more possible monitoring program may be performed based on an analysis of the patient's patient-specific factors to determine the most appropriate monitoring program that will not interfere with the patient's patient-specific factors and will most likely result in a positive engagement with the patient.

Once the engagement optimization engine generates an optimized PPEP for each patient, the engagement optimization engine outputs the PPEP to both the patient and the assessor according to the patient-specific factors and the program-specific factors (step 610) for implementation and monitoring of the PPCP. Thus, the engagement optimization engine takes into consideration not only the patient-specific factors of each of the patients but also the program-specific factors of the medical facility. The PPEP is then utilizes to engage with the patient and thereby improve healthcare provided to the patient and reduce subsequent costs of care.

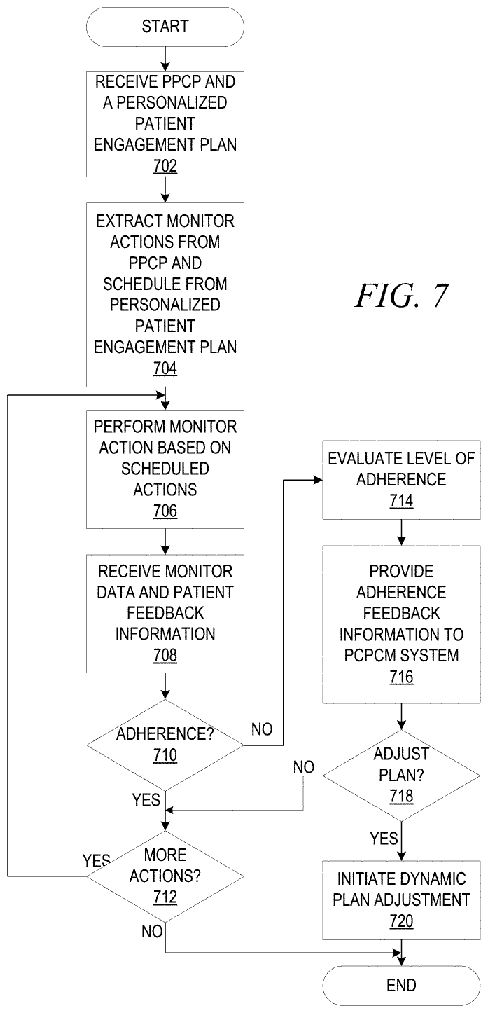

FIG. 7 is a flowchart outlining an example operation for monitoring a patient's performance with regard to a prescribed PPCP based on the personalized patient engagement plan (PPEP) in accordance with one illustrative embodiment. As shown in FIG. 7, the operation starts by receiving a PPCP and a PPEP (step 702). The monitor actions are the extracted from the PPCP and a schedule is extracted from the PPEP (step 704). A next monitor action in the schedule of monitor actions with regard to this patient is performed based on the schedule (step 706). It should be appreciated that the performance of such monitor actions may be automated, may be performed by a human, or may be a semi-automatic process in which different aspects of the monitor action are performed by an automated system and by a human.

In response to the monitor action being performed, monitor data and patient feedback information are received (step 708). For example, this may involve interrogating a health/activity monitoring device associated with the patient and receiving the corresponding data as a result. As another example, this may involve a human assessor calling the patient, asking the patient some questions about the patient's adherence to the PPCP, and then performing data entry to enter the monitor data and patient feedback information into the assessor system. In still another example, this may involve the patient logging onto an online system and inputting monitor data into the system which then reports the information to the assessor system, e.g., a patient entering blood sugar measurement data, weight data, symptom data, or the like. Many different ways of obtaining monitor data and patient feedback data may be utilized depending on the desired implementation of the illustrative embodiments.

Based on the monitor data and patient feedback information received, a determination is made by the assessor system as to whether the patient is adhering to the patient action required in the PPCP (step 710). If at step 710 the patient action in the PPCP is being adhered to, then a determination is made as to whether more patient actions in the PPCP to be checked (step 712). If at step 712 there are more actions to be checked, the operation returns to step 706. If at step 712 there are no more actions to be checked, then the operation terminates.

If at step 710 the patient action is not being adhered to, as may be determined from a comparison of the patient's monitor data and feedback to the requirements of the patient action in the PPCP, then an evaluation of the level of adherence is performed (step 714). Adherence feedback information is provided to the PCPCM system (step 716) and a determination is made as to whether the level of adherence is such that it warrants an adjustment of the patient actions in the PPCP (step 718). This determination may take into account various factors including the nature and importance of the patient action to the overall goal of the PPCP, e.g., taking medication may be considered much more important that walking for 30 minutes a day, a number of times this patient action has not been adhered to over a specified period of time, e.g., patient fails to walk for 30 minutes for 3 days in the past 5 days, an amount of the patient action that was actually achieved, e.g., the patient walked for 20 minutes but not 30 minutes, and the like. Based on a determined level of adherence and the nature and importance of the patient action, the assessor system determines whether an adjustment of the PPCP is needed (step 718).

If at step 718 an adjustment is needed, then the dynamic plan adjustment operations of the PCPCM system are initiated by a request from the assessor system (step 720) and the operation terminates. If at step 718 an adjustment is not needed, then the operation continues to step 712 where it is determined whether more patient actions in the PPCP need to be evaluated.

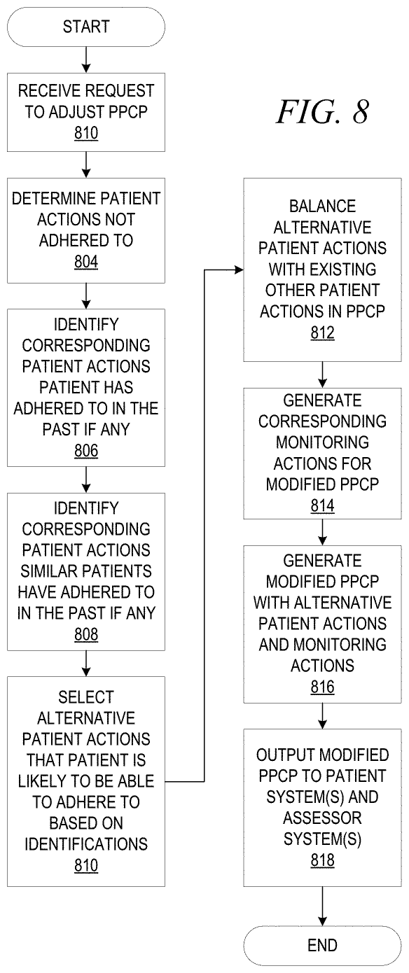

FIG. 8 is a flowchart outlining an example operation for adjusting a personalized patient health care plan based on an evaluation of a patient's adherence to a prescribed personalized patient health care plan in accordance with one illustrative embodiment. As shown in FIG. 8, the operation starts by receiving a request to adjust the PPCP for a patient, such as from the assessor system (step 802). The patient actions not adhered to are determined (step 804) and corresponding patient actions that the patient has adhered to in the past (if any) are identified (step 806). Corresponding patient actions in similar patient PPCPs that the similar patients have adhered to in the past are also identified (step 808).

Alternative patient actions that the patient is likely to be able to adhere to are selected based on the identification in steps 806 and 808 (step 810). The alternative patient actions are balanced with existing patient actions in the PPCP (step 812). This balancing may comprise adjusting other patient actions based on the alternative patient actions so as to achieve the same overall goals of the patient care plan, e.g., adjusting nutrition based patient actions based on changes to exercise or medication based patient actions.

Based on the modified patient actions, corresponding monitoring actions for the modified PPCP are generated (step 814) and a modified PPCP with the alternative patient actions and monitoring actions is generated (step 816). The modified PPCP is output to the patient system(s) and assessor system(s) (step 818) and the operation terminates.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for personalizing a patient care plan for a specific patient's own unique set of lifestyle characteristics such that the patient care plan is not generally applicable to a plurality of patients but is specific for the one patient. Information from various lifestyle information sources may be used along with patient care plan guidelines, demographic information, medical information, various resources, and the like, to generate a personalization of a more generic patient care plan that meets the desired goals for addressing a patient's medical condition. The personalization of the patient care plan may take into consideration patient actions that are successfully and unsuccessfully performed by the patient in other patient care plans, and by other similar patients with regard to their own PPCPs. This may be done on a historical basis as well.

Additionally, the mechanisms of the illustrative embodiments generate and implement a PPEP to monitor each patient's adherence to the personalized care plan as well as assist health professionals, assessors, automated assessment systems, and the like, in performing actions and initiating communications to maintain ongoing treatment and care of the patient. The mechanisms of the illustrative embodiments take into consideration not only the patient-specific factors of each of the patients but also the program-specific factors of the medical facility. In order to determine an optimal engagement plan for engaging with each patient based on the cohort associated with the patient and patient-specific factors, as well as program-specific factors for programs which may be applicable to the patient, the illustrative embodiments provide an engagement optimization engine may then be utilized to output the engagement plan and/or implement the engagement plan so as to engage with the patient and thereby improve healthcare provided to the patient and reduce subsequent costs of care.

Furthermore, the mechanisms of the illustrative embodiments provide monitoring actions for monitoring the patient's adherence to the PPCP and initiation of modifications to the PPCP when such adherence meets pre-defined criteria indicative of a need for a modification in the patient care plan.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems and Ethernet cards are just a few of the currently available types of network adapters.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and a memory comprising instructions which, when executed by the at least one processor, causes the at least one processor to implement an engagement optimization engine for generating and implementing a personalized patient engagement plan to monitor a patient's adherence to a personalized patient care plan, the method comprising:
receiving, by the engagement optimization engine, a cohort of patients with each patient in the cohort of patients having different personal characteristics;
for each patient in the cohort of patients, identifying, by the engagement optimization engine, a set of patient-specific factors;
identifying, by the engagement optimization engine, a set of program-specific factors associated with a set of programs available to the patient;
evaluating, by the engagement optimization engine, each set of patient-specific factors and the program-specific factors to generate the personalized patient engagement plan for each patient such that the personalized patient engagement plan meets the needs of the patient in view of the set of programs offered; and
outputting, by the engagement optimization engine, the personalized patient engagement plan for each patient to an assessor in order to implement monitoring of the personalized patient care plan generated for the patient.

2. The method of claim 1, wherein the patient-specific factors include one or more of a predicted risk level associated with the patient's medical condition, care gaps for the patient, socio-demographics associated with the patient, opt-ins and preferences indicated by the patient, or engagement history.

3. The method of claim 1, wherein the program-specific factors include one or more of available engagement programs for the patient, delivery costs for delivering the particular types of engagement programs, expected engagement program level, value of an expected outcome from the engagement program, or current capacity of the engagement program.

4. The method of claim 1, wherein the personalized patient care plan is generated by instructions causing the at least one processor to implement a personalized patient care plan (PPCP) system, the method comprising
obtaining, by the PPCP system, demographic and medical information about the patient;
automatically generating, by the PPCP system, an initial patient care plan for the patient, comprising a sequence of goals for the patient, based on an analysis of the obtained demographic and medical information for the patient;
obtaining, by the PPCP system, lifestyle information about the patient from a plurality of lifestyle information sources, wherein the lifestyle information characterizes a lifestyle of the patient;
modifying, by the PPCP system, the initial patient care plan to comprise one or more patient actions specific to the patient based on the lifestyle information and clinical best practices, thereby generating a personalized patient care plan; and
outputting, by the PPCP system, the personalized patient care plan to a patient computing device.

5. The method of claim 4, wherein modifying the initial patient care plan to comprise one or more patient actions specific to the patient based on the lifestyle information comprises:
categorizing, by the PPCP system, the patient in accordance with a plurality of lifestyle categories; and
modifying, by the PPCP system, at least one of the sequence of goals for the patient or one or more initial patient actions in the initial patient care plan, based on results of categorizing the patient in accordance with a plurality of lifestyle categories.

6. The method of claim 4, wherein modifying the initial patient care plan to comprise one or more patient actions specific to the patient based on the lifestyle information comprises, in response to analyzing the lifestyle information, at least one of:
scheduling, by the PPCP system, a patient action to be performed by the patient based on existing schedule information specified in the lifestyle information;
directing, by the PPCP system, a patient action to be performed at one or more specific locations associated with locations specified in the lifestyle information;
selecting, by the PPCP system, one or more foods to be consumed based on availability of foods to the patient, food preferences of the patient, and patient schedule information specified in the lifestyle information; or
selecting, by the PPCP system, a physical activity to be part of the one or more patient actions in response to analysis of the lifestyle information indicating availability of facilities for performing the selected physical activity.

7. The method of claim 4, wherein the personalized patient care plan comprises a personalized monitoring plan that comprises identifications of one or more monitoring actions, associated with corresponding patient actions in the personalized patient care plan, to be performed by the assessor that executes the one or more monitoring actions when assessing the patient's adherence to the personalized patient care plan.

8. A computer program product comprising a computer readable storage medium having a computer readable program stored therein, wherein the computer readable program, when executed on a computing device, causes the computing device to implement an engagement optimization engine for generating and implementing a personalized patient engagement plan to monitor a patient's adherence to a personalized patient care plan, and further causes the computing device to:
receive, by the engagement optimization engine, a cohort of patients with each patient in the cohort of patients having different personal characteristics;
for each patient in the cohort of patients, identify, by the engagement optimization engine, a set of patient-specific factors;
identify, by the engagement optimization engine, a set of program-specific factors associated with a set of programs available to the patient;
evaluate, by the engagement optimization engine, each set of patient-specific factors and the program-specific factors to generate the personalized patient engagement plan for each patient such that the personalized patient engagement plan meets the needs of the patient in view of the set of programs offered; and
output, by the engagement optimization engine, the personalized patient engagement plan for each patient to an assessor in order to implement monitoring of the personalized patient care plan generated for the patient.

9. The computer program product of claim 8, wherein the patient-specific factors include one or more of a predicted risk level associated with the patient's medical condition, care gaps for the patient, socio-demographics associated with the patient, opt-ins and preferences indicated by the patient, or engagement history.

10. The computer program product of claim 8, wherein the program-specific factors include one or more of available engagement programs for the patient, delivery costs for delivering the particular types of engagement programs, expected engagement program level, value of an expected outcome from the engagement program, or current capacity of the engagement program.

11. The computer program product of claim 8, wherein the personalized patient care plan is generated by the computer readable program causing the computing device to implement a personalized patient care plan (PPCP) system, and further causes the computing device to:
obtain, by the PPCP system, demographic and medical information about the patient;
automatically generate, by the PPCP system, an initial patient care plan for the patient, comprising a sequence of goals for the patient, based on an analysis of the obtained demographic and medical information for the patient;
obtain, by the PPCP system, lifestyle information about the patient from a plurality of lifestyle information sources, wherein the lifestyle information characterizes a lifestyle of the patient;
modify, by the PPCP system, the initial patient care plan to comprise one or more patient actions specific to the patient based on the lifestyle information and clinical best practices, thereby generating a personalized patient care plan; and
output, by the PPCP system, the personalized patient care plan to a patient computing device.

12. The computer program product of claim 11, wherein the computer readable program to modify the initial patient care plan to comprise one or more patient actions specific to the patient based on the lifestyle information further causes the computing device to:
categorize, by the PPCP system, the patient in accordance with a plurality of lifestyle categories; and
modify, by the PPCP system, at least one of the sequence of goals for the patient or one or more initial patient actions in the initial patient care plan, based on results of categorizing the patient in accordance with a plurality of lifestyle categories.

13. The computer program product of claim 11, wherein the computer readable program to modify the initial patient care plan to comprise one or more patient actions specific to the patient based on the lifestyle information further causes the computing device to, in response to analyzing the lifestyle information, perform at least one of:
schedule, by the PPCP system, a patient action to be performed by the patient based on existing schedule information specified in the lifestyle information;
direct, by the PPCP system, a patient action to be performed at one or more specific locations associated with locations specified in the lifestyle information;
select, by the PPCP system, one or more foods to be consumed based on availability of foods to the patient, food preferences of the patient, and patient schedule information specified in the lifestyle information; or
select, by the PPCP system, a physical activity to be part of the one or more patient actions in response to analysis of the lifestyle information indicating availability of facilities for performing the selected physical activity.

14. The computer program product of claim 11, wherein the personalized patient care plan comprises a personalized monitoring plan that comprises identifications of one or more monitoring actions, associated with corresponding patient actions in the personalized patient care plan, to be performed by the assessor that executes the one or more monitoring actions when assessing the patient's adherence to the personalized patient care plan.

15. An apparatus comprising:
at least one processor; and
at least one memory coupled to the at least one processor, wherein the at least one memory comprises instructions which, when executed by the at least one processor, cause the at least one processor to implement an engagement optimization engine for generating and implementing a personalized patient engagement plan to monitor a patient's adherence to a personalized patient care plan, and further cause the at least one processor to:
receive, by the engagement optimization engine, a cohort of patients with each patient in the cohort of patients having different personal characteristics;
for each patient in the cohort of patients, identify, by the engagement optimization engine, a set of patient-specific factors;
identify, by the engagement optimization engine, a set of program-specific factors associated with a set of programs available to the patient;
evaluate, by the engagement optimization engine, each set of patient-specific factors and the program-specific factors to generate the personalized patient engagement plan for each patient such that the personalized patient engagement plan meets the needs of the patient in view of the set of programs offered; and
output, by the engagement optimization engine, the personalized patient engagement plan for each patient to an assessor in order to implement monitoring of the personalized patient care plan generated for the patient.

16. The apparatus of claim 15, wherein the patient-specific factors include one or more of a predicted risk level associated with the patient's medical condition, care gaps for the patient, socio-demographics associated with the patient, opt-ins and preferences indicated by the patient, or engagement history.

17. The apparatus of claim 15, wherein the program-specific factors include one or more of available engagement programs for the patient, delivery costs for delivering the particular types of engagement programs, expected engagement program level, value of an expected outcome from the engagement program, or current capacity of the engagement program.

18. The apparatus of claim 15, wherein the personalized patient care plan is generated by the instructions further causing the at least one processor to implement a personalized patient care plan (PPCP) system, and further causing the at least one processor to:
obtain, by the PPCP system, demographic and medical information about the patient;
automatically generate, by the PPCP system, an initial patient care plan for the patient, comprising a sequence of goals for the patient, based on an analysis of the obtained demographic and medical information for the patient;
obtain, by the PPCP system, lifestyle information about the patient from a plurality of lifestyle information sources, wherein the lifestyle information characterizes a lifestyle of the patient;
modify, by the PPCP system, the initial patient care plan to comprise one or more patient actions specific to the patient based on the lifestyle information and clinical best practices, thereby generating a personalized patient care plan; and
output, by the PPCP system, the personalized patient care plan to a patient computing device.

19. The apparatus of claim 18, wherein the instructions to modify the initial patient care plan to comprise one or more patient actions specific to the patient based on the lifestyle information further cause the at least one processor to:
categorize, by the PPCP system, the patient in accordance with a plurality of lifestyle categories; and
modify, by the PPCP system, at least one of the sequence of goals for the patient or one or more initial patient actions in the initial patient care plan, based on results of categorizing the patient in accordance with a plurality of lifestyle categories.

20. The apparatus of claim 18, wherein the instructions to modify the initial patient care plan to comprise one or more patient actions specific to the patient based on the lifestyle information further cause the at least one processor to, in response to analyzing the lifestyle information, perform at least one of:
schedule, by the PPCP system, a patient action to be performed by the patient based on existing schedule information specified in the lifestyle information;
direct, by the PPCP system, a patient action to be performed at one or more specific locations associated with locations specified in the lifestyle information;
select, by the PPCP system, one or more foods to be consumed based on availability of foods to the patient, food preferences of the patient, and patient schedule information specified in the lifestyle information; or
select, by the PPCP system, a physical activity to be part of the one or more patient actions in response to analysis of the lifestyle information indicating availability of facilities for performing the selected physical activity.
